(19) United States
(12) Patent Application Publication
(10) Pub. No.: US 2012/0165717 A1
(43) Pub. Date: Jun. 28, 2012

(54) MEDICAL COMPRESSION PRODUCT, SYSTEM UTILIZING SUCH PRODUCT, AND PROGRAM FOR USE THEREWITH

(75) Inventor: Jawad Ameen Jawad Al Khaburi, PC Ruwi 112 (OM)

(73) Assignee: CONVATEC TECHNOLOGIES INC., Las Vegas, NV (US)

(21) Appl. No.: 13/048,339

(22) Filed: Mar. 15, 2011

Related U.S. Application Data
(60) Provisional application No. 61/426,093, filed on Dec. 22, 2010.

(51) Int. Cl. A61L 15/00; A61B 5/00

(52) U.S. Cl. 602/75; 600/300

(57) ABSTRACT

A medical compression product (“MCP”) for applying pressure to a limb of a patient may include one or more sensors integrally united therewith for measuring information indicative of the pressure applied by the MCP. The sensors may be permanently or removably attached to the MCP, and the sensors may be grouped into particular predetermined regions. The sensors may communicate (e.g., by use of wires or wirelessly) with a computer system that provides information to the user regarding the application of the MCP. The MCP may include bandages in the form of elongated fabric strips and tubular hosiery products. Wires connected to the sensors and communicating with the computer system may be aligned along or transverse to the longitudinal dimension of the bandage.

### CROSS-REFERENCE TO RELATED APPLICATIONS

This application claims the benefit of the filing date of U.S. Provisional Patent Application No. 61/426,093 filed Dec. 22, 2010, the disclosure of which is hereby incorporated herein by reference.

### BACKGROUND OF THE INVENTION

1. Field of the Invention

The present invention relates to a medical compression product, such as a bandage, for application to a limb of a patient for applying pressure to the limb, and also to a system including such a medical compression product and a program for use with such system.

2. Description of the Related Art

External compression applied using one of a variety of medical devices, collectively known as medical compression products (“MCP”), is the cornerstone of treatment for patients with venous disease and/or lymphoedema. MCP may include: extensible or non-extensible bandages (used with or without other interface materials); hosiery applied in one or more layers; orthostatic products (e.g., non-extensible sheets applied using a hook-and-loop fastening system); and pneumatic devices. The successful use of MCP may depend upon application of the products in a way that ensures that effective pressures (i.e., “interface pressures”) are applied between the product and the patient's skin.

One technique to assist in gauging whether the correct pressure has been applied by a bandage is through the use of geometric shapes on the bandage (e.g., ellipses or rectangles sewn or printed on the surface) that expand as the bandage is stretched during application. The geometric shapes are designed such that they distort to form a different shape (e.g., a circle or square, respectively) when a predetermined pressure or amount of extension has been applied. However, it can be difficult to determine at what point a geometric shape on the surface of the bandage has reached the target shape.

Another technique for indicating when the correct pressure is being applied includes providing two lines on the surface of a bandage that are spaced apart by a known distance. As the bandage is applied, the lines move apart due to the stretching of the material. The desired pressure may be indicated by a particular distance between the lines, which can be confirmed, for example, by comparison to spaced-apart marks on a reference card.

Alternatively, bandage manufacturers may simply recommend that the product be extended by a certain proportion (e.g., 50%) of its unstretched length. However, in practice, it can be difficult to estimate the required extension as a proportion of the unstretched length. Moreover, it can be difficult to maintain the desired amount of extension during the course of applying the entire bandage to the patient.

### BRIEF SUMMARY OF THE INVENTION

It would be desirable to provide easier to use and more accurate MCP, which are desirably capable of providing more information regarding the applied pressure.

One aspect of the present invention provides a medical compression product for application to a limb of a patient for applying pressure to the limb. A medical compression product according to this aspect of the invention may include a thin, flexible piece of material for wrapping at least partially around the limb and a sensor integrally united with the piece of material. The sensor may be operable to measure a predetermined parameter indicative of a pressure being applied by the piece of material to the limb of the patient.

According to one aspect of the invention, the medical compression product may include multiple sensors. According to this aspect of the invention, a subset of the sensors may be grouped together in a predetermined region of the piece of material. The predetermined region may correspond to a predetermined location on the limb of the patient.

According to another aspect of the invention, the medical compression product may be a compression bandage. According to yet another aspect of the invention, the sensor or sensors may be flexible. According to yet a further aspect of the invention, the piece of material may include an attachment structure for removably uniting each sensor with the piece of material.

According to further aspects of the invention, the medical compression product may include a transmission device integrally united with the piece of material. According to this aspect of the invention, the transmission device may be configured to transmit information regarding each sensor to a remote computer system. Such transmission may be performed wirelessly. In accordance with this aspect of the invention, the transmission device may include a radio-frequency identification (“RFID”) tag.

In accordance with another aspect of the invention, a medical compression system is provided. A medical compression system according to this aspect of the invention may include a processor, an output device, and a medical compression product, such as a medical compression product in accordance with one of the aforementioned aspects of the invention.

Still another aspect of the invention provides a non-transitory computer readable medium having stored thereon a program executable by a computer.

### BRIEF DESCRIPTION OF THE DRAWINGS

FIG. 1 is a simplified, schematic plan view depicting a compression bandage in accordance with one embodiment of the invention.

FIG. 2A is a simplified, schematic plan view depicting a compression bandage in accordance with a further embodiment of the invention.

FIG. 2B is a perspective view depicting a compression bandage in accordance with the embodiment of FIG. 2A.

FIG. 3 is a perspective view depicting a compression bandage in accordance with yet another embodiment of the invention.

FIG. 4A is a schematic view of a system including a compression bandage and a computer system in accordance with an embodiment of the invention.

FIG. 4B is a schematic view of a compression bandage in accordance with yet another embodiment of the invention.

FIG. 5 is a flow chart for a program run by a processor of the computer system of FIG. 4.

FIG. 6 is a display of an output device of the computer system of FIG. 4.

### DETAILED DESCRIPTION

The term “bandage,” as used herein, may encompass an elongated strip of flexible material (e.g., a fabric) for winding around a desired part of a patient's body. The term “bandage” may also encompass a tubular bandage (like a stocking), which has a predefined shape before application to a part of the body. The term “bandage,” however, is not limited to the above examples.

FIGS. 1-3 depict MCP 10 in accordance with several embodiments of the invention. The embodiments depicted in FIGS. 1 and 2A-B are compression bandages 12 shaped as elongated strips of flexible material, such as fabric (which may be extensible or non-extensible), and the embodiment of FIG. 3 is a tubular bandage 14 (i.e., hosiery product) shaped as a stocking for application to a patient's leg. The material of the bandages may be relatively thin. For example, the thickness of the bandage material may be between about 0.5 mm and about 2 mm. One exemplary thickness may be approximately 1 mm.

The MCP 10 may include a plurality of sensors 16 integrally united with the MCP 10. For example, the bandages shown in FIGS. 1-3 include one or more sensors 16 integrally united with the material (e.g., fabric) of the bandages. The present invention is not limited to bandages, however, and it is contemplated that sensors can be integrally united with other types of MCP as well (e.g., orthostatic devices).

The sensors 16 may be configured and arranged to measure the normal force and/or pressure applied to the skin of the patient by the applied MCP 10. The sensors may be flexible force and/or pressure sensors. Preferred types of sensors may include (but are not limited to): piezoelectric sensors; resistive or piezoresistive sensors (see, e.g., FLEXIFORCE® sensors manufactured by Tekscan, Inc. of Boston, Mass.; TACTILUS® sensors manufactured by Sensor Products Inc. of Madison, N.J.; and FSR® sensors manufactured by Interlink Electronics, Inc. of Camarillo, Calif.); sensors utilizing quantum tunneling composites (“QTCs”); and capacitive sensors (see, e.g., sensors manufactured by Pressure Profile Systems, Inc. of Los Angeles, Calif.; sensors manufactured by Novel GmbH of Munich, Germany; and sensors manufactured by Xsensor Technology Corporation of Calgary, Canada).

The sensors 16 may be as thin as possible (i.e., in the dimension normal to the skin surface), so that the thickness of the sensor 16 does not effectively modify the local curvature of the skin surface to a significant degree, which may decrease the accuracy of the measurement. For example, the thickness of the sensors 16 may be less than about 1 mm, and even less than about 0.5 mm. Additionally, the width of the sensor 16 (i.e., in a dimension parallel to the skin surface) may also be as small as possible, so that, particularly at highly curved locations of the skin surface, the curvature of the sensor 16 (based in part on its flexibility) does not effectively modify the local curvature of the skin surface and affect the accuracy of the measurement. A narrow sensor 16 may also provide a more precise measurement, as the sensor 16 may have a smaller area over which the pressures may be averaged. In one example, the width of each sensor 16 in its largest dimension may be less than or about equal to 14 mm. If the size of the sensors 16 does effectively modify the local curvature and results in an inaccurate measurement, a correction factor may be applied to the measured value to take into account the error introduced by the presence of the sensor 16.

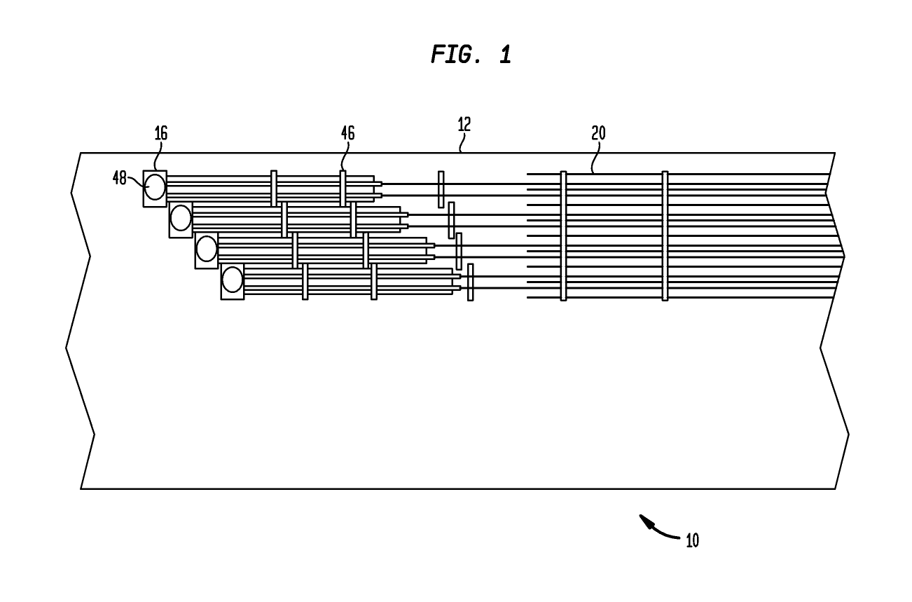

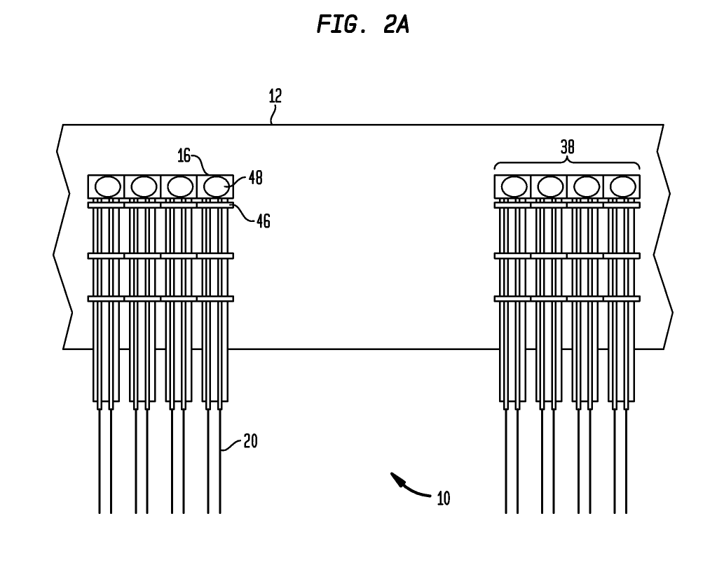

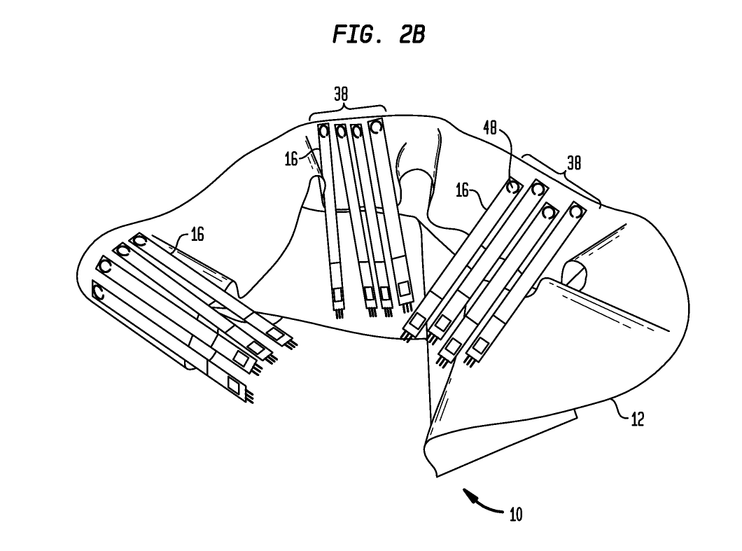

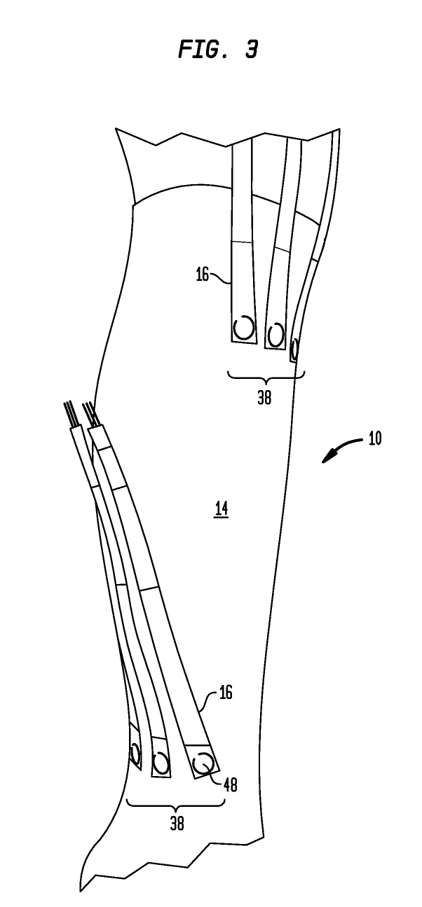

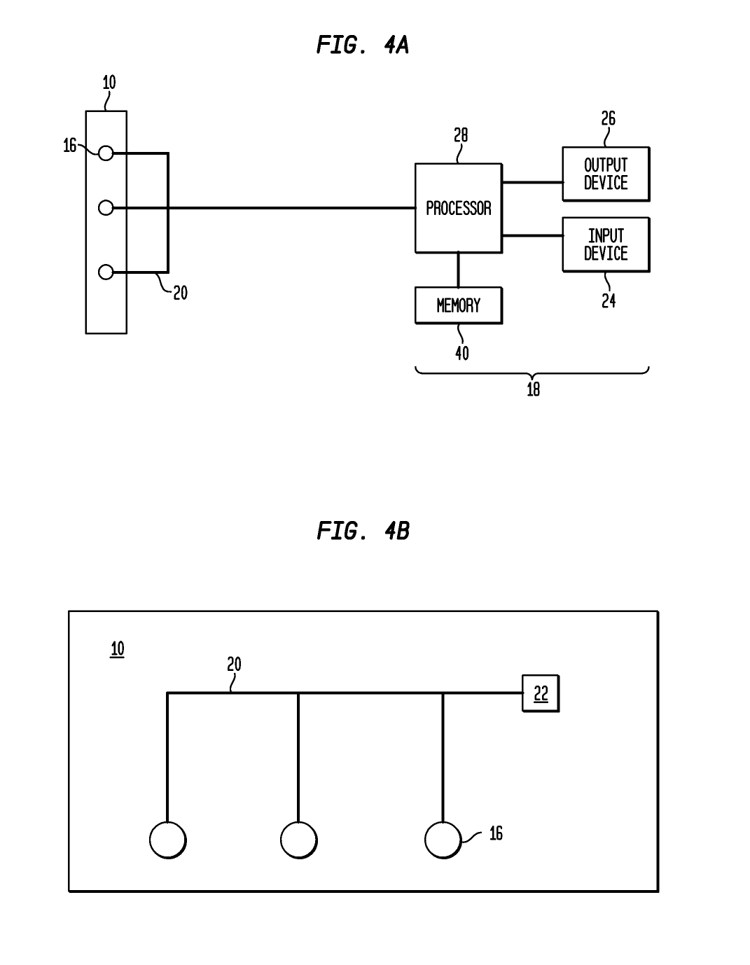

As shown in FIG. 4A, the sensors 16 may be connected to a computer system 18, which may be configured to receive information from the sensors 16 and communicate information to the user (e.g., through an output device 26). The sensors 16 may be connected to the computer system 18 by wires 20. The ends of the wires 20 opposite the sensors 16 may include connectors that are removably connectable to the computer system 18 (or to further electronic connections extending to the computer system 18), so that the MCP 10 can be disconnected from the computer system 18. In an alternative, as shown in FIG. 4B, the ends of the wires 20 opposite the sensors may be connected to one or more small devices 22 housed on the MCP 10 that can transmit information to a remote computer system 18. Each such small device 22 may wirelessly transmit such information to the computer system 18, and/or the small device may include a storage medium to store the collected information and may have one or more ports (not shown) to transmit such information to the computer system 18 when a connection is established between the small device and the computer system 18 via the port(s). The wireless transmission of the information may be accomplished using a variety of technologies, such as Bluetooth™, radio-frequency identification (“RFID”), ZigBee, etc. Beneficially, utilizing a technology such as ZigBee and, to an even greater extent, RFID may allow for relatively small and low cost components to be integrated with the MCP 10. For example, one or more RFID tags can be incorporated into the MCP 10 and connected to the sensors 16. Each RFID tag may be associated with one or more of the sensors 16. A separate reader (comparatively more expensive than the tags) can be used to communicate with the tag(s), in order to obtain the measurement information from the associated sensors 16. RFID tags may be “passive,” in which the electromagnetic waves from the reader power the circuitry of the RFID tag (by induction or wireless powering), and RFID tags may also be “active” or “semi-passive” (or “battery assisted passive”), both of which use an additional power source (e.g., a battery) to supply power to the tag. Any type of RFID tag may be incorporated into the MCP of the present invention. For example, in the case of a passive RFID tag, the power supplied to the tag by the reader may also be used to power the sensors 16. Alternatively, an additional power source may be used to provide power to the sensors 16.

Beneficially, the above types of wireless technologies may help keep the cost of the MCP 10 reasonably low, since the integrated components (e.g., sensors, wires, RFID tag) may be relatively inexpensive and may be disposed of with the MCP 10 at the end of its useful life. The above types of wireless components may also be relatively durable and able to withstand the conditions of use of the MCP 10.

The components of the computer system 18 may be separate or they may be integrated into a single device. In one example, those components may comprise a personal computer with associated input device or devices 24 (e.g., a keyboard) and output device or devices 26 (e.g., visual displays (such as monitors), audio devices (such as speakers), etc.). In an alternative, the computer system 18 may be integrated into a portable device, which may be small enough that it can move around with the patient. The computer system 18 may include a processor 28 configured to process the information from the sensors 16 and communicate the information to the output device(s) 26.

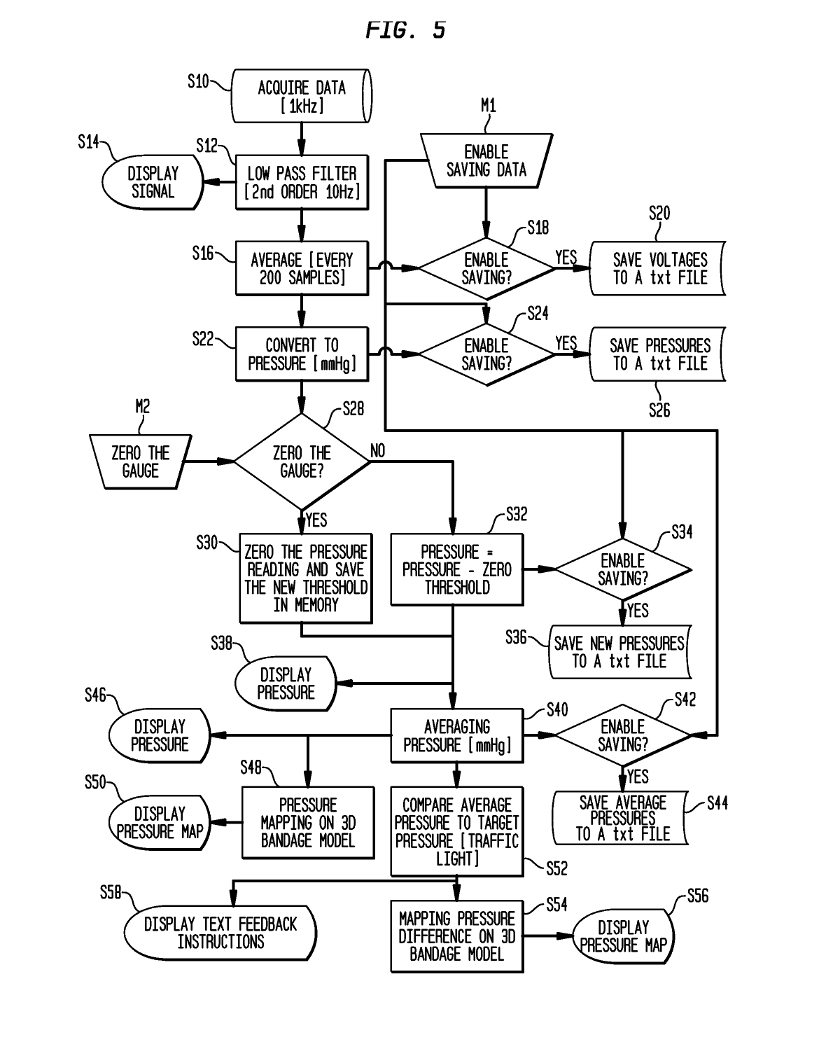

FIG. 5 illustrates an exemplary flow chart for a program which may be run by the processor 28 for acquiring data from the sensors 16 and displaying information via the output device 26. Such program may be supplied to the computer system 18 from an external source for immediate use thereat or for storage and subsequent use thereat, or, alternatively, may be stored in a non-transitory computer readable medium for use with the computer system 18. Such medium may include a computer disc, a hard disc drive, a read-only memory (“ROM”), a random-access memory (“RAM”), or other types of computer readable storage devices. As an example, such program may be stored in memory 40 of computer system 18. Additionally, it is noted that, although the steps of the program illustrated in the flow chart are shown in a particular order, a program in accordance with an embodiment of the invention may perform the steps in any desired order.

In step S10, data is acquired from the sensors 16 at a predetermined sampling frequency. That predetermined sampling frequency may be, for example, 1 kHz. The data may be in the form of voltages from the sensors 16. In step S12, the signal comprising the acquired data is passed through a filter, such as a low pass filter, which may remove noise from the signal. One exemplary low pass filter may be a second-order filter with a cutoff frequency of 10 Hz. The filtered signal is displayed by the output device 26 in step S14.

In step S16, the signal from the filter is averaged over a predetermined number of samples. For example, an average may be calculated for every 200 samples. In step S18, a determination is made as to whether the user has enabled data saving in manual operation Ml. If data saving has been enabled, the averaged data (e.g., average voltages) from step S16 is saved (e.g., to a file, such as a text file) in step S20. If data saving has not been enabled, the averaged data is not saved (not shown). In either situation, the processing proceeds (not shown) to step S22.

In step S22, the averaged data from step S16 is converted into pressure values (e.g., in mmHg). In step S24, a determination is made as to whether the user has enabled data saving. If data saving has been enabled, the pressure values are saved (e.g., to a text file) in step S26. If data saving has not been enabled, the pressure values are not saved (not shown). In either situation, the processing proceeds (not shown) to step S28.

In step S28, a determination is made as to whether the user is zeroing the gauge in manual operation M2. If the user is zeroing the gauge, the current pressure value from step S22 is saved in memory (such as memory 40 of computer system 18) as the zero threshold in step S30. If the user is not zeroing the gauge, new pressures are calculated in step S32. The new pressures equal the pressure values from step S22 minus the zero threshold saved in memory from step S30. In step S34, a determination is made as to whether the user has enabled data saving. If data saving has been enabled, the new pressure values are saved (e.g., to a text file) in step S36. If data saving has not been enabled, the new pressure values are not saved (not shown). In either situation, the processing proceeds (not shown) to step S38.

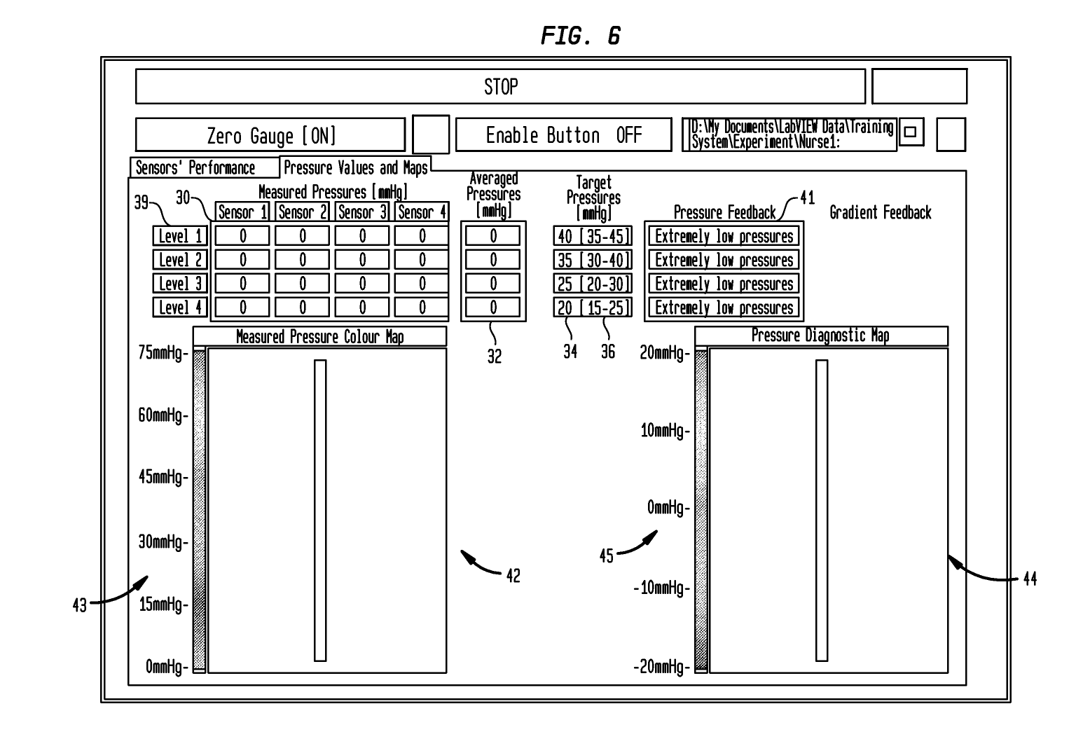

In step S38, the new pressures are displayed by the output device 26 (see, e.g., the numerical pressure values 30 for each of the sensors in FIG. 6). In step S40, for each predefined region of the bandage, the new pressure values calculated in step S32 from all of the sensors within the region are averaged. In step S42, a determination is made as to whether the user has enabled data saving. If data saving has been enabled, the average pressure values from step S40 are saved (e.g., to a text file) in step S44. If data saving has not been enabled, the average pressure values are not saved (not shown). In either situation, the processing proceeds (not shown) to step S46.

In step S46, the average pressure values from step S40 are displayed by the output device 26 (see, e.g., the average pressure values 32 in FIG. 6). In step S48, the average pressure values from step S40 are mapped onto a model of the MCP. For example, the average pressure values for the sensors within a particular region may be associated with the location of that region on a 3D model of the bandage. A graphical representation of this mapping is displayed by the output device 26 in step S50 (see, e.g., the color coded pressure map 42 in FIG. 6). The average pressure values from step S40 are also compared to target pressure values (or ranges of target pressure values) in S52. For example, the difference between an average regional pressure value and a target pressure value for that particular region may be calculated. That difference is then mapped onto a model of the MCP in step S54. That is, for example, the pressure differences with respect to each region may be associated with the respective locations of the regions on a 3D model of the bandage. A graphical representation of this mapping is displayed by the output device 26 in step S56 (see, e.g., the color coded pressure map 44 in FIG. 6).

In step S58, feedback information (such as textual instructions) is displayed by the output device 26. For example, if one of the average pressure values from step S40 is below a target pressure range (or beyond an acceptable deviation from a target pressure value), the output device 26 may display a textual message stating that the applied pressure is too low (see, e.g., the text signals 41 in FIG. 6). One example of an acceptable deviation from a target pressure value is a deviation of up to 5 mmHg from the target pressure value. The feedback information displayed by the output device 26 in step S58 may provide qualitative information regarding the pressure differences calculated in step S52, as discussed further below.

One example of a display provided by the output device 26 is illustrated in FIG. 6. The output device 26 may display numerical values for the interface pressures. For example, a numerical value 30 may be displayed for each of the sensors 16, and/or an average value 32 from multiple sensors 16 may be displayed. The multiple sensors 16 for which an average value 32 is displayed may be a group 38 of sensors 16 representing a region of the patient's limb (e.g., all sensors 16 at a particular level 39 of the limb, as shown in FIGS. 2B, 3, and 6). The output device 26 may also display target pressure values 34 and/or ranges 36 of desirable pressures. The computer system 18 may also calculate and display numerical values representing the difference between the pressure actually applied and the target pressure values and/or ranges. The target pressure values 34 and/or ranges 36 may be based on various factors, including the type of venous disease being treated and the type of limb being treated. The computer system 18 may be configured to store data (e.g., in memory 40) relating to such factors, such that target pressure values 34 and/or ranges 36 can be retrieved and/or calculated based on the selected factors. The computer system 18 may be configured to receive such factors from the user (e.g., via input device 24). The computer system 18 may also be configured to receive the target pressure values 34 and/or ranges 36 directly from the user.

The output device 26 may be configured to display non-numerical information (e.g., qualitative information) regarding the pressure value or values. Such information may be provided in connection with each of the sensors 16 and/or in connection with a group (such as group 38) of sensors 16. In one example, the non-numerical information may include color-coded outputs representing variance of the applied pressure from the target pressure values and/or ranges. For example, the colors may be: white for pressures at least 20 mmHg (˜2700 Pa) higher than the target pressure value; red for pressures at least 10 mmHg (˜1300 Pa) higher than the target pressure value; green for no difference (or within an acceptable range (e.g., 5 mmHg)) from the target pressure value; light blue for pressures at least 10 mmHg lower than the target pressure value; and pink for pressures at least 20 mmHg lower than the target pressure value. In another example, non-numerical text signals or indications 41 (and/or audio signals) (e.g., “very low,” “slightly low,” “correct,” “slightly high,” and “very high”) may be provided for use by an operator. In yet another example, only three non-numerical signals (e.g., text and/or color) may be used: one for the correct pressure value, one for higher pressure values, and one for lower pressure values. Although examples having five and three non-numerical signals have been discussed, additional embodiments may provide more or fewer non-numerical signals to indicate different degrees of pressure deviations.

The output device 26 may provide a map that graphically illustrates the MCP 10 and/or the body part being treated. Such a map may indicate (e.g., by color codes, as discussed above) the pressure values and/or deviations in different regions, so that the carer can visualize the consistency of application of the MCP (e.g., bandage). As shown in FIG. 6, one color coded map 42 may illustrate the qualitative pressure values across a bandage, and another color coded map 44 may illustrate the qualitative deviations from the target pressures across the bandage. The output device 26 may also include color codes 43, 45 next to the respective maps 42, 44 that indicate the colors associated with particular pressure values. It is to be appreciated that, although the maps 42, 44 and associated color codes 43, 45 are not illustrated in FIG. 6 in the aforementioned colors, those items could be provided with color-coded outputs as discussed above, or any other desired colors.

Beneficially, in the case where a pressure gradient is to be applied upon the limb, the non-numerical information communicated to the user may be consistent at different locations along the limb, even when the target pressure values 34 are not consistent at those locations. That is, if the target pressure value is 20 mmHg at one location and 40 mmHg at another location, the output device 26 may display information relative to the target pressure value 34 at each location. For example, if the target pressure value is 20 mmHg and the user is applying 10 mmHg, the output device 26 may indicate that the applied pressure is less than the target amount (and/or may provide qualitative and/or quantitative information indicating the degree to which the applied pressure is less than the target amount). In a location where the target pressure value 34 is, for example, 40 mmHg and the user is applying 30 mmHg, the output device 26 may provide similar information. This consistent form of feedback may help make it easier for the user to apply the MCP 10 properly, as the user may not need to keep track of different target pressure values at different locations along the limb, and the non-numerical information may be easier to interpret during use than numerical values.

The information displayed by the output device 26 may allow the carer to make adjustments to the MCP 10 as necessary. The MCP 10 and computer system 18 may be configured to provide real-time feedback during the application of the MCP 10, which may allow the carer to make adjustments as the MCP 10 is being applied. Such real-time feedback may also be useful as a training device, to help a trainee learn how to properly apply a MCP 10 (such as an elongated compression bandage 12) with the correct pressure and extension. Use of a wireless component, as described above, may be particularly helpful, as there may be no wires to interfere with the application of the MCP 10.

The information processed by the computer system 18 can be collected and/or monitored continuously or periodically over an extended period of time, in order to inform the carer about changes in the interface pressure delivered by the MCP 10. This may help the carer understand the changes in interface pressure that may have taken place because of changes in, for example, the limb size and shape, as commonly happens during compression therapy. Such information may be collected over the course of a patient management session with a carer or over a longer period of time, and such collected information may allow the carer to make judgments regarding reapplication of the MCP 10. For example, the removable connections discussed above may be periodically connected to a computer or other device to remotely transmit the collected data (or the wireless connection may periodically or continuously transmit the data) to a carer, or to the electronic record-keeping and/or monitoring systems associated with the carer.

The MCP 10 may also interface with other electronic systems. For example, the MCP 10 may be configured to communicate data to an electronic medical record system, which may be located at a remote location. The MCP 10 may also be configured to interface with a system having a printing function, so that hard copy reports of the information collected by the MCP 10 may be generated.

The sensors 16 and wires 20 may be united with the MCP 10 so as to form an integral unit. The sensors 16 may be secured at particular locations on the MCP 10. For example, the sensors may be arranged into several groups 38 that, when the MCP 10 is applied to the patient, correspond to particular portions of the body where pressure sensing is desirable. In one example, where the MCP 10 is applied to a patient's leg, the sensors 16 may be grouped into ankle, gaiter, mid-calf, below knee regions, and so forth. It may be desirable to apply different pressures at different locations along the MCP 10 (e.g., a gradient extending along the length of the MCP 10). For example, in the case of a leg, it may be desirable to apply a pressure of about 40 mmHg (˜5300 Pa) at the ankle, decreasing to about 20 mmHg (˜2700 Pa) below the knee. In the case where a pressure gradient is desirable, the computer system 18 may be programmed to associate each of the sensor regions with a different desired pressure value.

In one example, as shown in FIGS. 1-2B, threads 46 (made of, e.g., elastic or inelastic lycra, cotton, or nylon) may be spaced or arranged along the sensors 16 and wires 20 to attach those components to the bandage 12. For example, the threads 46 may be formed into loops that receive portions of the sensors 16 and wires 20. The sensors 16 and wires 20 may be detachable from the loops, so that the bandage 12 can be washed for repeated uses. The loops may maintain their shape and position so that, after the bandage 12 is washed, the sensors can be easily reattached to the predetermined portions of the bandage 12.

The sensors 16 and wires 20 may be attached to the bandage 12 by other means, however. Preferably, such securing means will be configured to flex with the bandage and will not interfere with the sensors' ability to take measurements. In one example, the electronic components may be removably attached to the bandage 12 by a hook-and-loop fastening system. The sensors 16 and wires 20 need not necessarily be removable from the bandage 12, however. Providing sensors 16 and wires 20 that can withstand washing may allow those electrical components to be more completely integrated into the bandage 12 (e.g., by securely sewing the electronic components into the material of the bandage 12).

Uniting the sensors 16 with the MCP material is believed to have numerous benefits. For example, the MCP 10 will desirably be an integrated, self-contained product. Such a product is likely quicker and easier to apply than separately applying an MCP 10 and an electronic sensing system. In this regard, if the sensors 16 are to be provided before the MCP 10 is applied, for example, so that the sensors 16 are disposed between the MCP 10 and the patient's skin, it may be difficult to place the sensors 16 on the patient's body in such a way that they remain in the desired positions and are not disturbed by the application of the MCP 10. Additionally, integrating the sensors 16 with the MCP 10 allows the sensors 16 to be arranged in predetermined, desirable locations with respect to the MCP 10. This may reduce the need for the carer to independently determine the best locations for the sensors 16 and then attach separate sensors 16 to those locations. Also, integrating the sensors 16 with the MCP 10, rather than separately applying the sensors 16 before applying the MCP 10, may reduce the shear stress applied to the sensors by the MCP 10. This reduction in shear stress may reduce the measurement error of the sensors 16 and extend the lifetime of the sensors 16.

In the case of a compression bandage shaped as an elongated strip, the wires may extend along the longitudinal dimension of the bandage 12, as shown in FIG. 1, or the wires may extend substantially transverse to the longitudinal dimension, as shown in FIGS. 2A-B.

The longitudinally-arranged structure depicted in FIG. 1 can provide numerous benefits. For example, the wires are all contained within the boundaries of the bandage 12, which may reduce the clutter of having multiple, independent wires 20 emanating from different portions of the bandage 12. If the bandage 12 is extensible, the wires 20 may be configured to slide with respect to the bandage 12 or otherwise accommodate such stretching. Alternatively, instead of the bandage 12 including wires 20, particular threads of the bandage 12 may be formed from an electrically conductive material, so as to form conductive paths extending along the bandage 12 to the sensors 16. In another alternative, conductive paths may be formed from a coating (e.g., an electrically conductive paint-like material) applied to a surface of the bandage 12 and extending from each of the sensors 16. These alternatives to wires (i.e., conductive threads or coating) may be utilized in any of the embodiments of the invention.

There may be several issues associated with the arrangement of FIG. 1, however. For example, since the wires 20 run along the longitudinal dimension of the bandage 12, relatively long wires 20 may be needed. Additionally, since the wires 20 extend along much of the bandage 12, there may be less available space to position sensors 16, which may limit the number of sensors 16 that can be provided (e.g., four sensors 16 are illustrated in FIG. 1). The proximity of the wires 20 to the sensors 16 may also cause errors in some of the sensors 16 that are interspersed with the longitudinally arranged wires 20. Both the sensors 16 and the wires 20 may also need to be removed before washing the bandage 12.

The embodiment depicted in FIGS. 2A-B may improve on some or all of these issues. For example, since the wires 20 no longer extend along the longitudinal dimension of the bandage 12, shorter wires 20 may be used, and it may also not be necessary to have the wires 20 slide with respect to the bandage 12 when the bandage 12 is stretched. Additionally, since the wires 20 are generally outside the boundary of the bandage 12, only the sensors 16 (and not the wires 20) may need to be removed in order to wash the bandage 12. Furthermore, the arrangement of FIGS. 2A-B may allow the sensors 16 to be pre-connected to the bandage 12, and the wires 20 may be connected to the sensors 16 after the bandage 12 is fully applied.

The arrangement of FIGS. 2A-B may also allow a greater number of sensors 16 to be provided, as a greater portion of the bandage 12 will be available to receive sensors 16 (and not covered by wires 20). In one embodiment, sixteen sensors 16 may be used. As shown in FIGS. 2A-B, the sensors 16 may be arranged in groups 38 of sensors 16 (e.g., groups of four sensors). Each group 38 of sensors 16 may be arranged to associate with a different region of the body (e.g., ankle, gaiter, mid-calf, and below knee regions of the leg). In an embodiment of the computer system 18 in which an average pressure value from multiple sensors 16 within a particular region is displayed (as discussed above), the values from the sensors 16 in a particular group 38 may be averaged to yield an average pressure value for the corresponding region. As shown in FIG. 2B, the sensing portion 48 of the sensors 16 may be attached to the top half of the bandage 12. This arrangement may insure that the sensors 16 report the pressure applied by both layers of the bandage 12 when the bandage 12 is applied with a spiral overlap of a predetermined amount, such as a 50% overlap.

The tubular bandage 14 depicted in FIG. 3 has a predefined shape before it is applied to a part of the patient's body. This may beneficially lead to more accurate final positioning of the sensors 16, as the final sensor locations will be less likely to depend on the manner in which the bandage is applied. After the tubular bandage 14 is positioned on the patient's body, an elongated compression bandage may be applied on top of the tubular bandage 14. The tubular bandage 14 may be constructed to itself apply some pressure to the patient's body independent of an elongated bandage wrapped around it. The sensors 16 in the embodiment of FIG. 3 may be attached to the inside or the outside surface of the tubular bandage 14. If the sensors 16 are located on the inside surface of the tubular bandage 14, the sensors 16 will be able to measure the pressure applied by both the tubular bandage 14 and by any elongated compression bandages wrapped over the tubular bandage 14, rather than just the pressure applied by the elongated compression bandage.

Another benefit of the tubular bandage 14 depicted in FIG. 3 is that it may be relatively easy to obtain real-time measurements during the application of an elongated compression bandage. That is, the sensors 16 of the tubular bandage 14 can be connected to the computer system 18 before an elongated compression bandage is applied. Then, as the elongated compression bandage is wrapped around the tubular bandage 14, the output device 26 of the computer system 18 may communicate real-time information regarding the applied pressures. Although such real-time measurements may also be obtained with the embodiments depicted in FIGS. 1-2B, having the wires 20 from the bandages 12 pre-connected to computer system 18 may make it more difficult to properly apply the bandage 12. If such real-time measurements are desired, it may be preferred to use a wireless-type device in connection with the embodiments of FIGS. 1-2B.

The systems and apparatuses shown and described herein may be used in conjunction with any or all of the systems and apparatuses shown and described in the pending U.S. nonprovisional patent application filed on the same date and naming the same inventor as the present nonprovisional patent application, and entitled “Training System For Applying A Medical Compression Product, And A Device And Program For Use Therewith,” the entire disclosure of which is fully incorporated by reference herein.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A medical compression product for application to a limb of a patient for applying pressure to the limb, said medical compression product comprising:
a thin, flexible piece of material for wrapping at least partially around the limb; and
a sensor integrally united with said piece of material, said sensor being operable to measure a predetermined parameter indicative of a pressure being applied by said piece of material to the limb of the patient.

2. The medical compression product of claim 1, comprising a plurality of sensors.

3. The medical compression product of claim 2, wherein a subset of the plurality of sensors are grouped together in a predetermined region of the piece of material, said predetermined region corresponding to a predetermined location on the limb of the patient.

4. The medical compression product of claim 1, wherein said pressure is a pressure applied normal to a surface of the limb of the patient.

5. The medical compression product of claim 1, wherein the medical compression product is a compression bandage.

6. The medical compression product of claim 1, wherein said piece of material is an elongated strip having a longitudinal dimension.

7. The medical compression product of claim 6, wherein said sensor is connected to a wire for transmitting electrical signals between said sensor and an external device, said wire being aligned along the longitudinal dimension.

8. The medical compression product of claim 6, wherein said sensor is connected to a wire for transmitting electrical signals between said sensor and an external device, said wire being aligned substantially transverse to the longitudinal dimension.

9. The medical compression product of claim 1, wherein said sensor is detachable from said piece of material.

10. The medical compression product of claim 1, wherein said piece of material includes an attachment structure for removably uniting said sensor with said piece of material.

11. The medical compression product of claim 10, wherein said attachment structure comprises at least one loop of thread for surrounding and securing at least a portion of said sensor.

12. The medical compression product of claim 1, wherein said sensor is flexible.

13. The medical compression product of claim 1, further comprising a transmission device integrally united with said piece of material, said transmission device being configured to transmit information regarding sensor to a remote computer system.

14. The medical compression product of claim 13, wherein said transmission device is configured to wirelessly transmit the information regarding said sensor to the remote computer system.

15. The medical compression product of claim 14, wherein said transmission device includes a radio-frequency identification (RFID) tag.

16. A medical compression system, comprising:
a medical compression product for application to a limb of a patient for applying pressure to the limb, said medical compression product including:
(i) a thin, flexible piece of material for wrapping at least partially around the limb; and
(ii) a sensor integrally united with said piece of material, said sensor being operable to measure a predetermined parameter indicative of a pressure being applied by said piece of material to the limb of the patient;
a processor arranged to receive data from the sensor corresponding to the measured predetermined parameter; and
an output device connectable to the processor to provide information to an operator relating to the measured predetermined parameter.

17. The system of claim 16, wherein said medical compression product includes a plurality of sensors arranged to provide measurements with respect to a first location and a second location on the limb; wherein said processor is arranged to compare the data received from the plurality of sensors to a plurality of numerical target pressure ranges which include a first target pressure range relating to the first location on the limb and a second pressure range relating to the second location on the limb, the second target pressure range having a different range of values than the first target pressure range; and wherein said information provided by said output device includes: (i) providing a non-numerical indication with respect to the first location when the pressure applied by the medical compression product to the limb at the first location is within the first target pressure range, and (ii) providing the same non-numerical indication with respect to the second location as with respect to the first location when the pressure applied by the medical compression product to the limb at the second location is within the second target pressure range.

18. The system of claim 16, wherein said medical compression product is a compression bandage.

19. The system of claim 16, further comprising a transmission device integrally united with the piece of material of said medical compression product, said transmission device being configured to transmit the data from the sensor corresponding to the measured predetermined parameter to a remote computer system including said processor.

20. A non-transitory computer readable medium having stored thereon a program executable by a computer, said program comprising:
comparing data received from a plurality of sensors in a medical compression product to a plurality of numerical target pressure ranges, the data corresponding to a predetermined parameter measured by the sensors, the predetermined parameter indicative of a pressure being applied by the medical compression product to a first location and a second location on a limb of a patient, wherein the target pressure ranges include a first target pressure range relating to the first location on the limb and a second target pressure range relating to the second location on the limb, the second target pressure range having a different range of values than the first target pressure range;
providing to an output device a first signal representative of a first non-numerical indication with respect to the first location when the pressure applied by the medical compression product to the limb at the first location is within the first target pressure range; and
providing to the output device a second signal representative of a second non-numerical indication with respect to the second location, the second non-numerical indication being the same as the first non-numerical indication when the pressure applied by the medical compression product to the limb at the second location is within the second target pressure range.
